(19) United States
(12) Patent Application Publication
(10) Pub. No.: US 2022/0143139 A1
(43) Pub. Date: May 12, 2022

(54) Modulating Neuroinflammation

(71) Applicant: The Schepens Eye Research Institute, Inc., Boston, MA (US)

(72) Inventors: Dong Feng Chen, Newtonville, MA (US); Kin-Sang Cho, Winchester, MA (US); Min Ji, Boston, MA (US)

(21) Appl. No.: 17/435,947

(22) PCT Filed: Mar. 3, 2020
(86) PCT No.: PCT/US2020/020794

§ 371 Date: Sep. 2, 2021

Related U.S. Application Data
(60) Provisional application No. 62/813,556, filed on Mar. 4, 2019.

(51) Int. Cl. A61K 38/17; A61P 27/02; A61P 27/06

(52) U.S. Cl. CPC A61K 38/1754; A61P 27/06; A61K 38/1709; A61P 27/02

(57) ABSTRACT

Compositions and methods for treating and/or reducing risk of development or progression of neuroinflammation and neurodegeneration, comprising HSP60 e.g., for nasal administration, and IGFBPL1, e.g., for nasal, systemic, or ocular, e.g., intravitreal, administration.

### CLAIM OF PRIORITY

This application claims the benefit of U.S. Provisional Patent Application Ser. No. 62/813,556, filed on Mar. 4, 2019. The entire contents of the foregoing are hereby incorporated by reference.

### RESEARCH OR DEVELOPMENT

This invention was made with Government support under Grant No. EY025259 awarded by the National Institutes of Health. The Government has certain rights in the invention.

### TECHNICAL FIELD

Described herein are compositions and methods for treating and/or reducing risk of development or progression of neuroinflammation and neurodegeneration, comprising HSP60 and/or HSP27, e.g., for nasal administration, and IGFBPL1, e.g., for nasal, systemic, or ocular, e.g., intravitreal, administration.

### BACKGROUND

Glaucoma is a leading cause of blindness and a globally unmet medical challenge.

### SUMMARY

Provided herein are methods for treating and/or reducing risk of development or progression of neuroinflammation and neurodegeneration in a subject in need thereof. The methods include administration of therapeutically effective amounts of one or more of: (i) HSP60 and/or HSP27, or active fragments thereof and/or (ii) IGFBPL or active fragments thereof, to the subject.

In some embodiments, the therapeutically effective amounts are sufficient to reduce inflammation and neuronal cell death in the subject.

In some embodiments, the methods include nasal administration of HSP60 and/or HSP27 and systemic (e.g., nasal or oral) or ocular administration of IGFBPL.

In some embodiments, ocular administration of IGFBPL comprises intravitreal injection.

In some embodiments, the subject has Glaucoma, Autism, Multiple Sclerosis, Alzheimer's Disease, Parkinson's Disease, Ischemic Retinopathy, Age-Related Macular Degeneration, Stroke, Ischemic and Traumatic Optic Neuropathy, or Diabetic Retinopathy.

In some embodiments, the method reduces inflammation and neuronal death in an eye of the subject.

In some embodiments, the method reduces inflammation and neuronal death in the brain or spinal cord of the subject.

Also provided herein are kits comprising a composition comprising HSP60 and/or HSP27, and a composition comprising IGFBPL1, for use in a method described herein.

Further, provided herein are compositions comprising HSP60 and/or HSP27, and/or a composition comprising IGFBPL1, for use in a method of treating and/or reducing risk of development or progression of neuroinflammation and neurodegeneration.

In some embodiments, the HSP60 and/or HSP27 is formulated for nasal administration and the IGFBPL1 is formulated for ocular, e.g., intravitreal administration. In some embodiments, the HSP60 and/or HSP27 is formulated for nasal administration and the IGFBPL1 is formulated for systemic, e.g., oral or nasal, administration. In some embodiments, the HSP60 and/or HSP27 is formulated for nasal administration and the IGFBPL1 is formulated for nasal administration. In some embodiments, one, two, or all three of the HSP60 and/or HSP27 and the IGFBPL1 are formulated together for nasal administration in a single composition.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

The patent or application file contains at least one drawing executed in color. Copies of this patent or patent application publication with color drawing(s) will be provided by the Office upon request and payment of the necessary fee.

FIG. 1. Microbead (MB)-induced elevation of intraocular pressure (IOP) not affected by treatment of heat shock protein (HSP) 60 via nasal spray. Left panel: Saline and MB-filled anterior chambers of mouse eyes. Right panel: IOP levels over time in non-treated saline injected control mice (light grey), saline-treated MB-injected mice (gray), and HSP60-treated MB-injected mice (black).

FIG. 2. Induction of Treg cells in mice received nasal spray of HSP60. Top panel: Live gating of CD4+ cells from the eye's draining lymph nodes, the superior cervical lymph nodes. Bottom panel: Histogram of Treg+ counts (CD4+ CD25+ FOXP3+) at 2 weeks post MB-injection comparing HSP60-treated and saline-treated mice.

FIGS. 3A-C. Rescue of vision by HSP60 nasal spray in MB-induced glaucoma mice. A-C. Visual contrast sensitivity (CS, 3A), Visual acuity (VA, 3B), and amplitudes (3C) of positive scotopic threshold response (pSTR) assessed at 2, 4 and 6 weeks post IOP elevation in all groups ***P<0.001 **P<0.01 *P<0.05.

FIGS. 4A-B. Rescue of retinal ganglion cells (RGCs) and axons by HSP60 nasal spray in MB-induced glaucoma mice. A-B. Quantification of retinal ganglion cells (RGCS; immunohistochemistry with Brn3a, 4A) and axons (4B) at 2, 4 and 6 weeks post-IOP elevation in all groups ***P<0.001 **P<0.01 *P<0.05 FIG. 5. Schematic of hypothetical mechanism of HSP60 nasal spray.

FIGS. 6A-D. IGFBPL1 protected RGCs against elevated IOP-induced damage and prevents the loss of RGC function and vision in a glaucoma model. A. IOP levels in sham- (triangles) or microbeads-injected mice received intravitreal injection of saline (squares) or IGFBPL1 (circles). B. RGC counts in naïve and sham-injected mice and MB-injected received saline or IGFBPL1 treatment. C. Quantification of ERG positive scotopic threshold response (pSTR) in Saline and IGFBPL1 treated mice, showing significantly improved RGC functions in IGFBPL1-treated mice compared to saline-treated mice. D. OKR tests showing significantly improved visual acuity (VA) and visual contrast sensitivity (CS) in IGFBPL1-treated compared to Saline-treated group when determined at 2-8 weeks post MB-injection.

FIGS. 7A-B. Microglial expression of IGFBPL1, IGF-1 receptor (IGF-1R) and IGF-1. A. Double-immunolabeling of microglia for microglia marker Iba-1 (green) and IGFBPL1 (red) in culture and in retinal whole-mounts that were counter-stained with a nuclear marker DAPI (blue). B. Double-immunolabeling of microglia marker Iba-1 (green) and IGF-1R (red) or IGF-1 (red) in the flat-mounts of adult mouse retinas.

FIGS. 8A-B. IGFBPL1 suppression of microglia activation in the glaucomatous mouse retina. A. Counts of activated microglia in retinal wholemounts of naïve (normal), microbeads-plus saline-injected (MB+Saline), and microbeads-plus IGFBPL1-injected (MB+IGFBPL1) mice at 5 to 14 days after IOP elevation. B. Results of qPCR showing induction of activated microglial markers following elevated IOP and suppression of activated microglial markers by IGFBPL1 administration.

FIG. 9. IGFBPL1 inhibits proinflammatory cytokine production inglaucomatous retina. Results of qPCR showing induction of activated proinflammatory cytokines following elevated IOP and their suppression by IGFBPL1 administration.

FIGS. 10A-B. IGFBPL1 deficiency lead to microglia activation in the adult retina. A. Quantification of activated microglia in retinal whole mounts. B. Result of qPCR showing increased levels of activated microglial cell markers in IBKO retina compared to WT retina.

FIG. 11. Progressive RGC loss in IGFBPL1 deficient mice. Quantification of RGC densities showing gradual loss of RGCs from 4 week- to 7 month-old IBKO mice.

FIG. 12. IGFBPL1 suppression of LPS-induced inflammation. Quantification results of proinflammatory cytokine levels in isolated microglial cultures treated with control, IGFBPL1, LPS and LPS+IGFBPL1.

FIG. 13. Protection of RGCs against ischemic reperfusion-induced death and functional damage. Data are expressed as the mean±SD (n=5 mice per group). * p<0.05 by student t-test.

### DETAILED DESCRIPTION

Described herein are methods for treating and reducing risk of development or progression of neuroinflammation and neurodegeneration. The methods include one or more of: (i) administration of HSP60 or HSP27, or active fragments thereof and/or (ii) administration of IGFBPL or active fragments thereof. The methods can be used to reduce inflammation and neuronal cell death in the eye and elsewhere, including the CNS and PNS. The methods can include nasal administration of HSP60 or HSP27 and nasal, systemic, or ocular administration of IGFBPL, e.g., intravitreal injection or ocular topical, e.g., for the treatment of glaucoma.

HSP60 (HSPD1, Heat Shock Protein Family D (Hsp60) Member 1)

Emerging evidence implicates an autoimmune mechanism in glaucoma, but its relative importance in disease pathogenesis has not yet been proven. As shown herein, neuron and vision loss in glaucoma, and other immune-related conditions, is associated with pre-existing memory T cells that are pre-sensitized by exposure to bacterial HSP60 in early life. Uncovering the immune mechanism and its association with commensal microbes in progressive neurodegeneration in glaucoma provides a basis for new diagnoses treatments.

The amino acid sequence of human hsp60 is provided in GenBank Accession Number NP 002147.2, and that of bacterial hsp60 is provided in GenBank Accession Number WP 000729117.1, incorporated herein by reference. The HSP60 is preferably formulated for nasal administration, to induce tolerance to HSP60. Alternatively, oral or subcutaneous administration can be used. See also WO2012118863.HSP27 (heat shock protein family B (small) member 1 (HSPB1))

HSP27, also known as HSPB1, is shown herein to directly induce pro-inflammatory responses of HMC3 cells. The amino acid sequence of human HSP27 is provided in GenBank Accession Number NP_001531.1, incorporated herein by reference. The HSP27 is preferably formulated for nasal administration, to induce tolerance to HSP27. Alternatively, oral or subcutaneous administration can be used. See also WO2012118863.

IGFBPL (Insulin Like Growth Factor Binding Protein Like 1)

Insulin growth factor binding protein like 1 (IGFBPL1) promotes survival and neurite outgrowth of neonatal mouse retinal ganglion cells (RGC) regulated via insulin like growth factor 1 mediated signaling pathways (Guo et al., Sci Rep. 2018 Feb. 1; 8(1):2054). As shown herein, IGFBPL1 is active in suppressing neuroinflammatory microglia in adult/post-neonatal animals. The amino acid sequence of human IGFBPL1 is provided in GenBank Accession Number NP 001007564, incorporated herein by reference. See also WO2012118796.

Methods of Treatment and Prevention

The compositions described herein can be administered to a subject to treat or prevent disorders associated with an abnormal or unwanted immune response, e.g., an neuroinflammatory or neurodegenerative disorder associated with excessive or to aberrant activation of microglia. Examples of such disorders include, but are not limited to, Non-Arteritic Ischemic Optic Neuropathy (NAION), Autism, Multiple Sclerosis, Alzheimer's Disease, Parkinson's Disease, Ischemic Retinopathy, Glaucoma, Age-Related Macular Degeneration, Stroke, Ischemic and Traumatic Optic Neuropathy, and Diabetic Retinopathy; in some embodiments, the disease is associated with vision loss and/or increased intraocular pressure. The methods can be used to treat subjects with those diseases, e.g., to reduce the risk of or treat vision and neuron loss associated with those diseases. See also U.S. Pat. No. 8/198,284; WO/2017/213504; WO2012118863; and WO2012118796, all of which are incorporated by reference herein.

The methods of treatment or prevention described herein can include administering to a subject a nasal or subcutaneous HSP60 or HSP27 composition, e.g., sufficient to stimulate the mucosal immune system. In some embodiments, the methods include administering a nasal HSP60 or HSP27 composition sufficient to increase levels of regulatory T cells, e.g., by about 50%, 75%, 100%, 200%, 300% or more over baseline.

In some embodiments, the methods include administering a nasal or subcutaneous HSP60 or HSP27 composition and/or an IGFBPL1 composition in amounts sufficient to produce an improvement in one or more clinical markers of vision loss (e.g., reduction in visual acuity) or of disability; for example, in multiple sclerosis, such markers could include gadolinium-enhancing lesions visualized by MRI, or Paty's, Fazekas' or Barkhofs MRI criteria, or McDonald's diagnostic criteria. The IGFBPL1 composition can be administered nasally, systemically, or ocular, e.g., by eye drops or intravitreal administration.

In some embodiments, the treatment is administered to a subject who has been diagnosed with a disorder associated with microglial activation; such a diagnosis can be made by a skilled practitioner using known methods and ordinary skill. In some embodiments, the methods include a step of diagnosing or identifying or selecting a subject with a disorder associated with microglial activation, or identifying or selecting a subject based on the presence or a diagnosis of a disorder associated with microglial activation. In some embodiments, the subject is an adult human, e.g., a human who is at least 18 years old, or is a post-neonatal human, e.g., who is at least 6 month or 1 year old.

Pharmaceutical Compositions and Methods of Administration

The methods described herein include the use of pharmaceutical compositions comprising one or more of HSP60, HSP27, or IGFBPL1 as an active ingredient.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration.

Pharmaceutical compositions are typically formulated to be compatible with its intended route of administration.

Examples of routes of administration include systemic parenteral, e.g., intravenous, intraperitoneal, intradermal, or subcutaneous; local to the eye, e.g., topical, intravitreal, intraocular, intraorbital, periorbital, subconjuctival, subretinal, subtenons or transscleral; and systemic oral administration. In some embodiments, intraocular administration or administration by eye drops, ointments, creams, gels, or lotions may be used, inter alia. In some embodiments, the composition is administered systemically, e.g., orally; in preferred embodiments, the composition is administered to the eye, e.g., via topical (eye drops, lotions, or ointments) administration, or by local injection, e.g., periocular or intravitreal injection; see, e.g., Gaudana et al., AAPS J. 12(3):348-360 (2010); Fischer et al., Eur J Ophthalmol. 21 Suppl 6:S20-6 (2011). Administration may be provided as a periodic bolus (for example, intravitreally or intravenously) or as continuous infusion from an internal reservoir (for example, from an implant disposed at an intra- or extra-ocular location (see, U.S. Pat. Nos. 5,443,505 and 5,766,242)) or from an external reservoir (for example, from an intravenous bag, or a contact lens slow release formulation system). The composition may be administered locally, for example, by continuous release from a sustained release drug delivery device immobilized to an inner wall of the eye or via targeted transscleral controlled release into the choroid (see, for example, PCT/US00/00207, PCT/US02/14279, PCT/US2004/004625, Ambati et al. (2000) Invest. Ophthalmol. Vis. Sci. 41:1181-1185, and Ambati et al (2000) Invest. Ophthalmol. Vis. Sci. 41:1186-1191). A variety of devices suitable for administering agents locally to the inside of the eye are known in the art. See, for example, U.S. Pat. Nos. 6,251,090, 6,299,895, 6,416,777, 6,413,540, and 6,375,972, and PCT/US00/28187.

Pharmaceutical compositions are typically formulated to be compatible with its intended route of administration. Examples of routes of administration include systemic (e.g., parenteral, nasal, subcutaneous, and oral) and local (ocular, e.g., intravitreal or topical ocular) administration. Thus also within the scope of the present disclosure are compositions comprising the compositions described herein in a formulation for administration for the eye, e.g., in eye drops, lotions, creams, e.g., comprising microcapsules, microemulsions, or nanoparticles. Methods of formulating suitable pharmaceutical compositions for ocular delivery are known in the art, see, e.g., Losa et al., Pharmaceutical Research 10:1(80-87 (1993); Gasco et al., J. Pharma Biomed Anal., 7(4):433-439 (1989); Fischer et al., Eur J Ophthalmol. 21 Suppl 6:S20-6 (2011); and Tangri and Khurana, Intl J Res Pharma Biomed Sci., 2(4):1541-1442 (2011).

General methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. The pharmaceutical compositions can also be prepared in the form of suppositories (e.g., with conventional suppository bases such as cocoa butter and other glycerides) or retention enemas for rectal delivery.

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

Kits

Also provided herein are kits for use in the methods described herein. For example, the kits can include a composition comprising HSP60 or HSP27, e.g., for nasal administration, and a composition comprising IGFBPL1, e.g., for nasal, systemic (e.g., oral) or ocular (e.g., topical ocular or intravitreal) administration. Instructions for use can also be included in the kits.

### EXAMPLES

The invention is further described in the following examples, which do not limit the scope of the invention described in the claims.

### Example 1. Immune Tolerance to HSP60 Attenuates Neurodegeneration in a Mouse Model of Glaucoma

Primary open angle glaucoma (POAG), a leading cause for blindness in the world, is a disease that damages the optic nerve. Although POAG has pre-dominantly been associated with high intraocular pressures (IOPs), therapies that influence IOP do not fully prevent vision loss and blindness.^{1,2}

Elevated intraocular pressure (IOP) induced T cell-mediated autoimmune responses to HSP60, and HSP-specific T-cell responses and neuronal loss are abolished after elevated IOP in mice raised in the absence of microflora.³⁻⁹ This example tested whether induction of tolerance to HSP60 would attenuate glaucomatous damage.

Immune tolerance to HSP60 was induced in 6-8 week old male and female C57BL/6J mice by administration of a low dose of HSP60 into the nostrils (2 uM of HSP60, daily for 7 days). Control mice were treated with saline. Glaucoma was induced by two injections of microbeads (MB) into the anterior chamber, maintaining IOP elevation for 8 weeks. IOP was monitored weekly. Visual function was assessed by optokinetic motor response (OMR) and electroretinogram scotopic threshold response (pSTR). Mice were sacrificed at 2, 4 and 8 weeks. Immune responses and T cell tolerance to HSP60 were analyzed by fluorence-activated cell sorting (FACS). Glaucomatous neural damage was quantified by retinal ganglion cell (RGC) and axon counts.

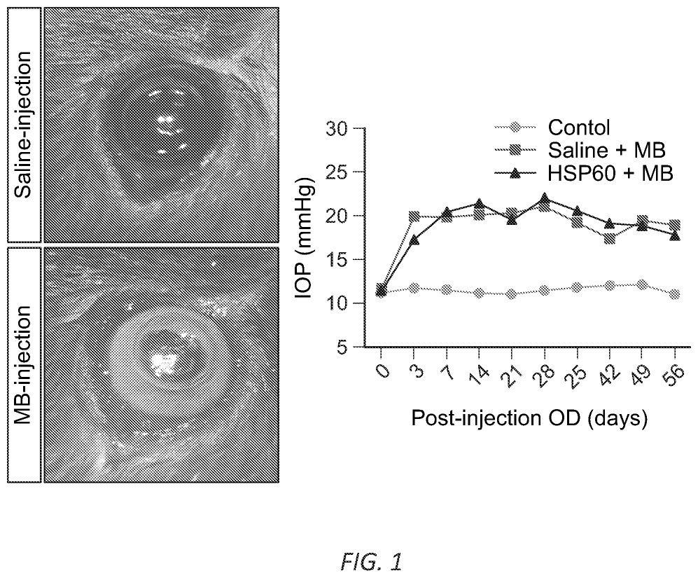

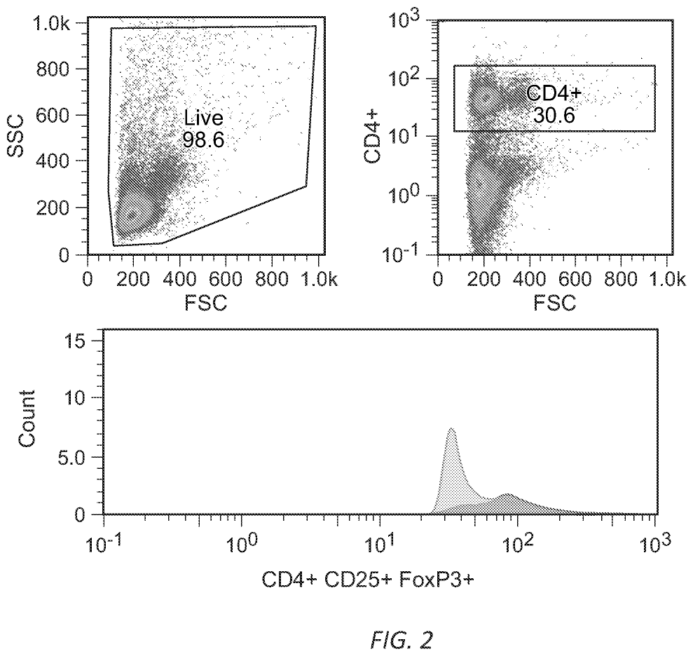

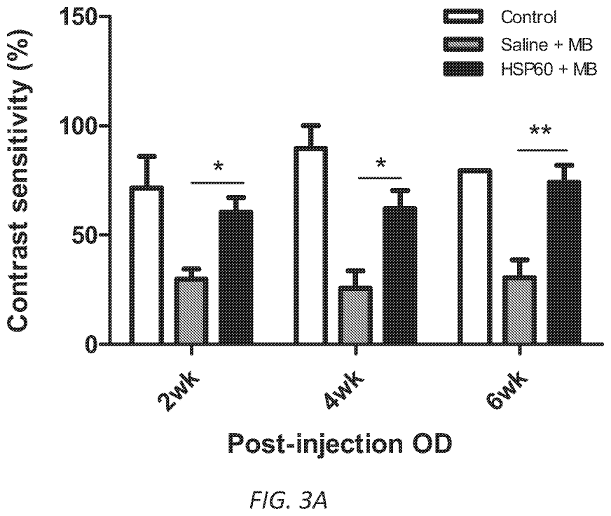

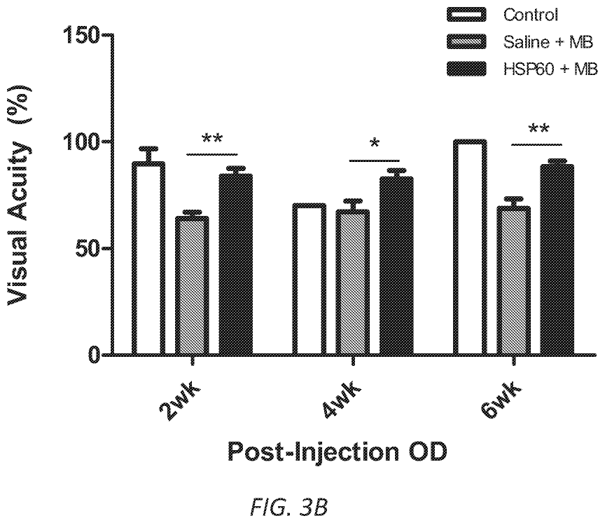

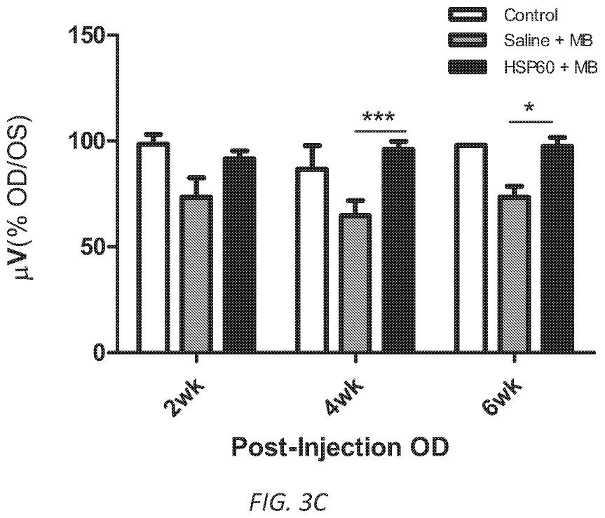

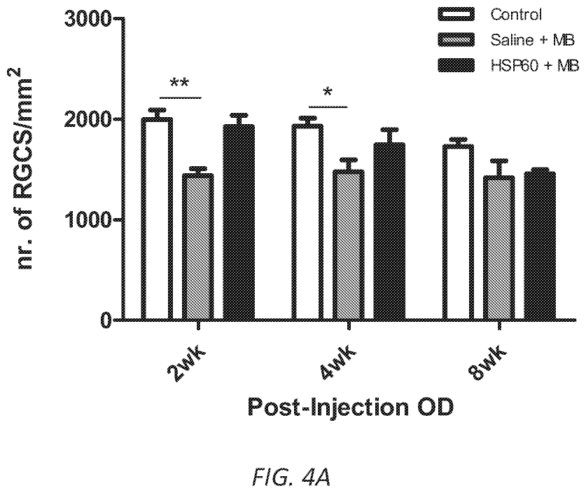

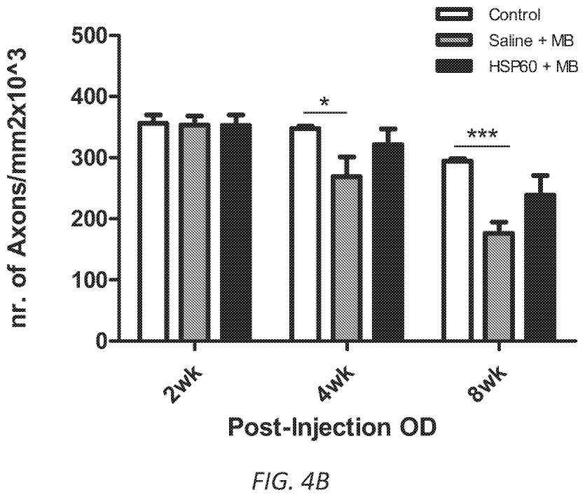

By 4 weeks, MB-injected eyes displayed an IOP of 20.1±0.56 mmHg or above compared to 11.6±0.21 mmHg in contralateral non-injected eyes (FIG. 1). Nostril administration of low dose HSP60 induced immune tolerance and increased levels of Treg as shown by FACS analysis (FIG. 2). We observed no significant differences in IOP levels between HSP60 or saline-treated mice. Treatment with HSP60 did not alter visual acuity (VA), contrast sensitivity (CS) or pSTR prior to MB-injection, as compared to saline-treated mice. However, HSP60-treated mice exhibited significantly higher VA and CS as assessed by OMR than saline-treated mice at all time-points after MB-injection (FIGS. 3A-C). Consistently, RGC function as assessed by pSTR was also significantly improved in HSP60 treated mice compared to saline-treated non-immune tolerated mice at all time-points after MB-injection (FIGS. 4A-B).

These results show that immune tolerance to HSP60 attenuates glaucomatous RGC loss and functional loss in mice, as shown in FIG. 5.

### Example 2. A Role for Insulin-Like Growth Factor Binding Protein Like Protein 1 in Microglia in Adult Mice

Insulin-like growth factor binding protein like protein 1 (IGFBPL1) plays a vital role in promoting axon growth and survival of retinal ganglion cell (RGC) during development (Guo et al., Sci Rep. 2018 Feb. 1; 8(1):2054). This requires the presence of insulin-like growth factor 1 (IGF1) and is mediated through IGF1 receptor (IGF1R). Since adult RGCs are known to express a low or undetectable level of IGF1R, this example explored whether IGFBPL1 supports RGC survival after injury in the adult retina.

Expression of IGF1, IGFBPL1 and IGF1R in the adult retina was examined in retinal whole-mounts using immunohistochemistry. The retinal whole-mounts were double-immunolabeled with primary antibody against an RGC marker Brn-3, or microglial marker Iba-1 to identify cell type-specific expression of IGF1, IGF1R and IGFBPL1.

Co-cultures of purified microglia and RGCs isolated from postnatal day 5 Cx3CR1/GFP mice were performed, either in the presence or absence of LPS and/or IGFBPL1 and IGF1. Neuronal survival was determined by Live/Dead Viability kit, and the percentage of live RGCs was quantified using Image J software.

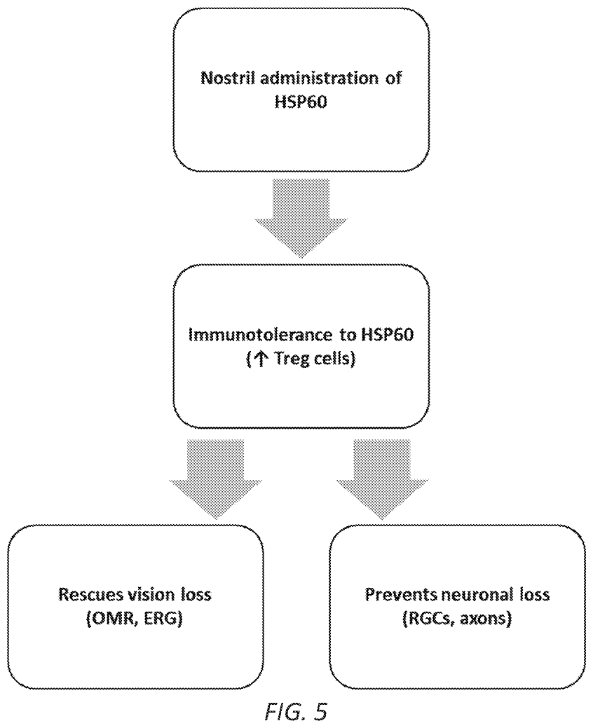

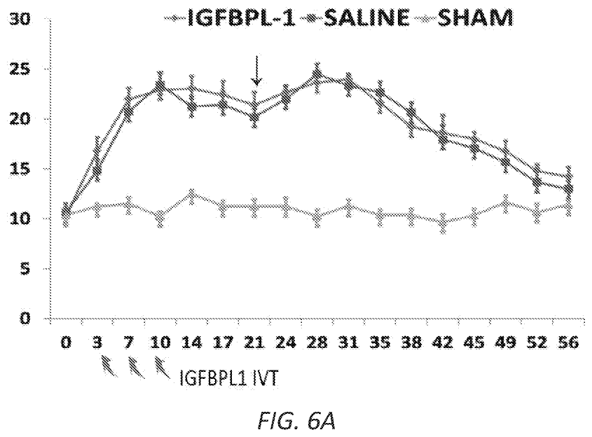

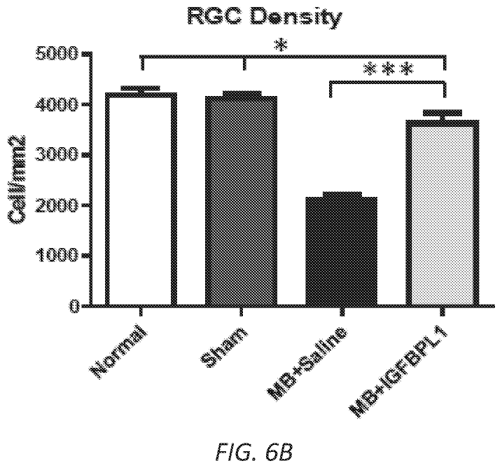

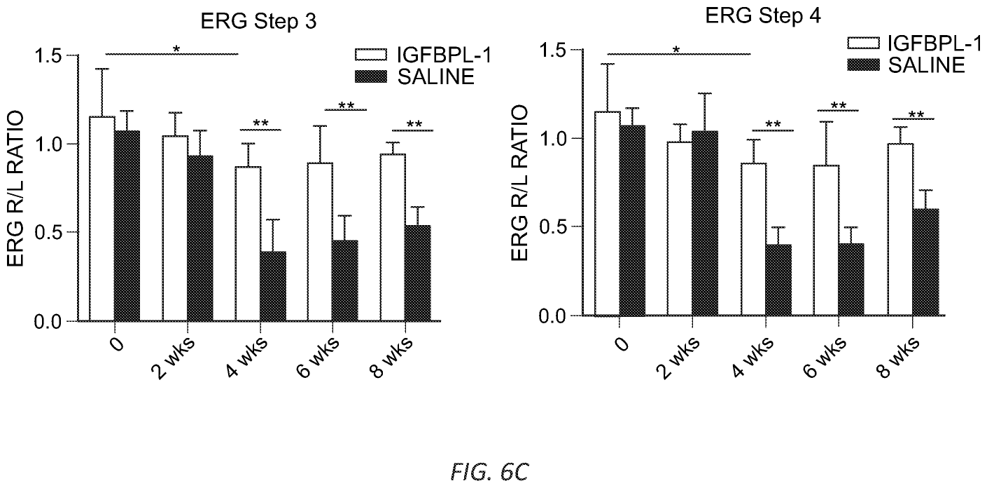

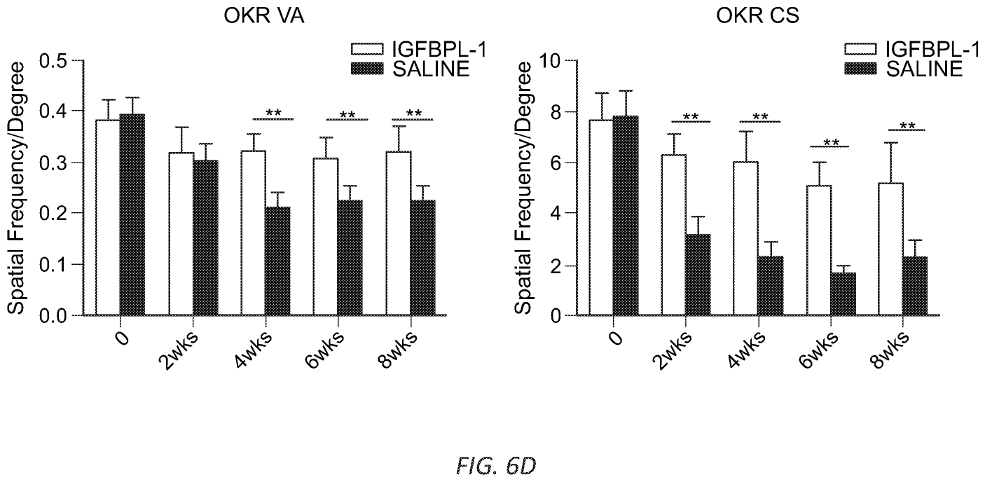

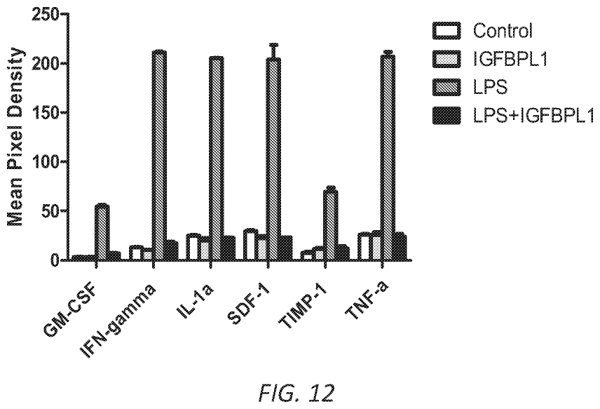

The results showed that IGF1, IGF1R, and IGFBPL1 were expressed by microglia, but not RGCs, in adult mouse retina (FIGS. 6A-B). Addition of IGF1 and/or IGFBPL1 to purified RGC cultures did not promote neuron survival, but LPS stimulated microglia activation and caused significant RGC death compared to the control cultures (P<0.05). IGF1 and/or IGFBPL1 significantly attenuated the neuronal cell death in LPS-treated microglia-induced RGC death in microglia-RGC co-cultures (See FIG. 12).

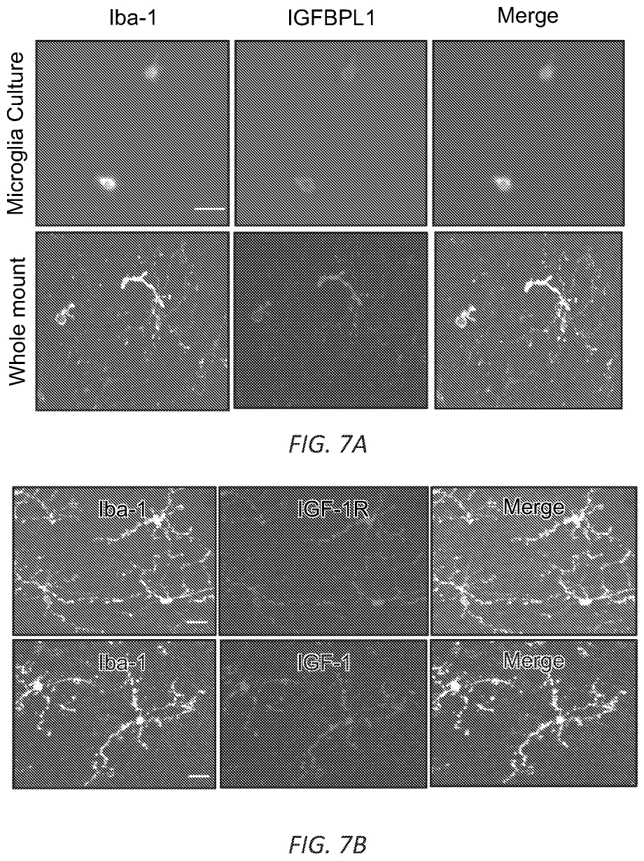

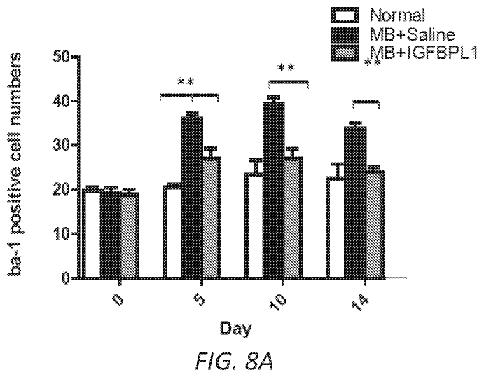

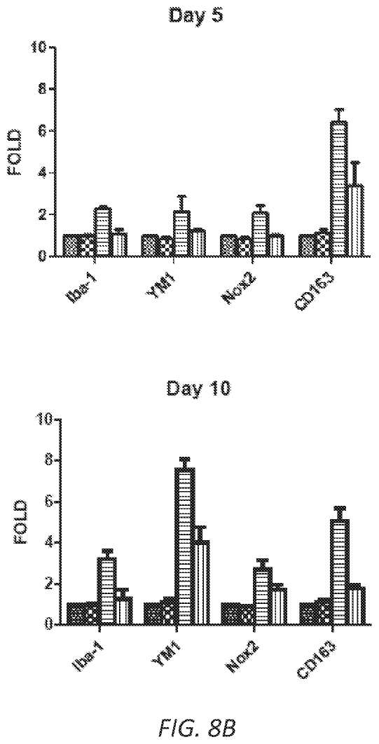

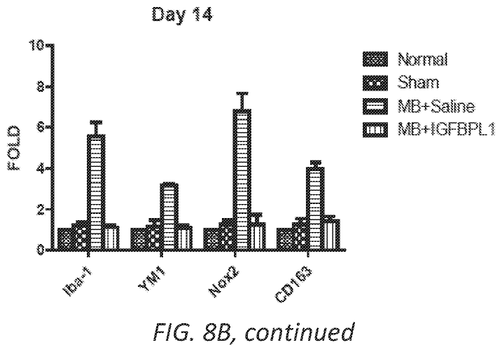

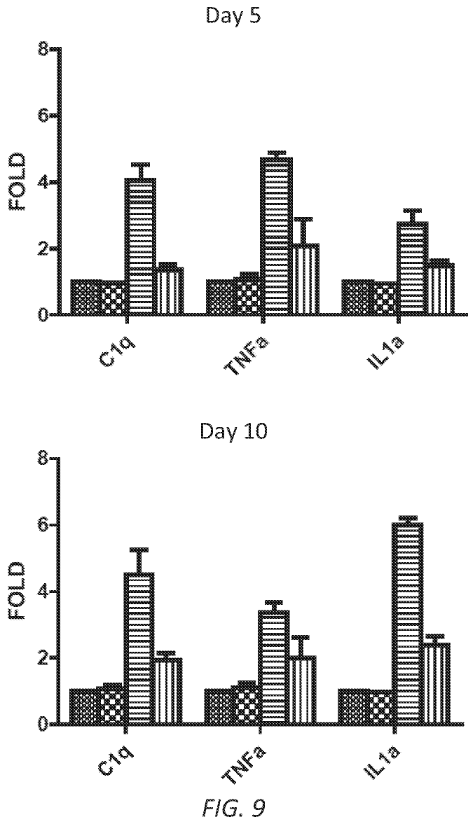

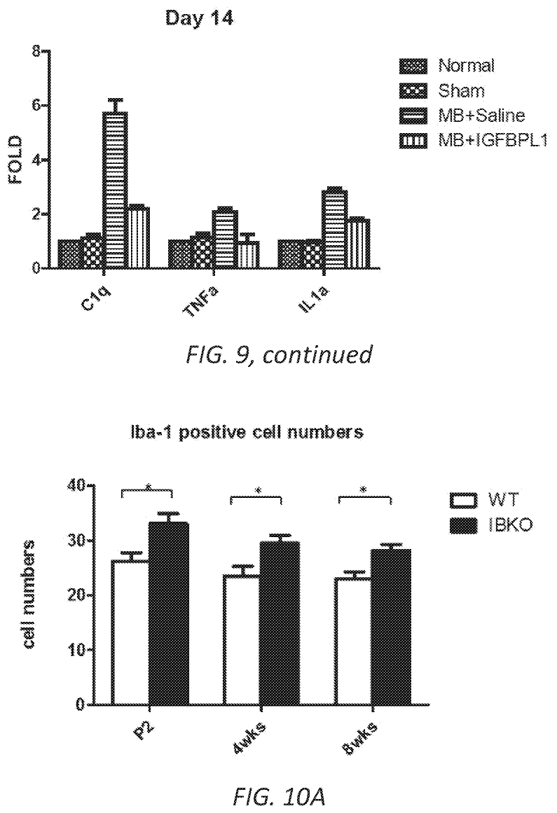

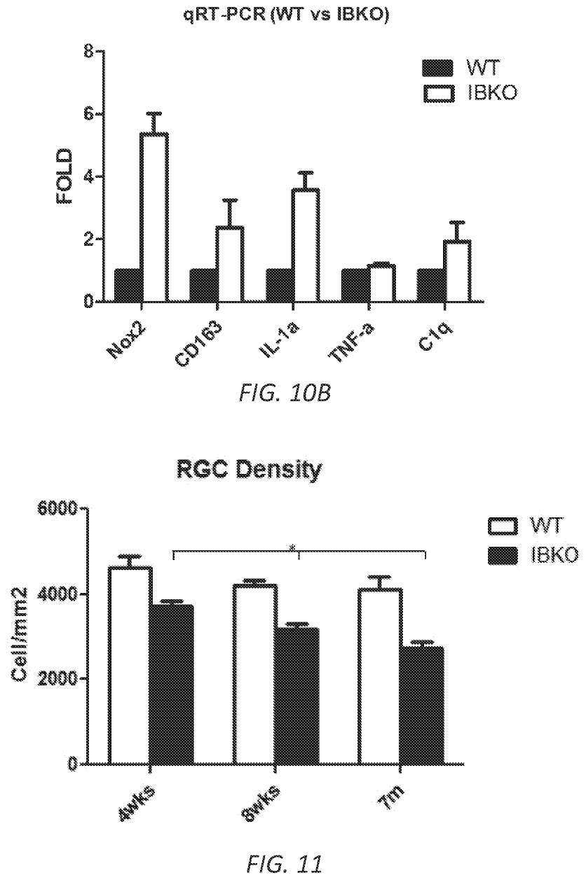

Intravitreal injection of IGFBPL1 at 3 and 10 days post IOP elevation protected RGCs against elevated IOP-induced damage and prevented the loss of RGC function and vision in a glaucoma model (FIGS. 7A-D). IGFBPL1 suppresses microglia activation in glaucomatous retina (FIG. 8A-B) and also inhibited microglial activation, reactive gliosis, and proinflammatory cytokine production (FIG. 9). As shown in FIGS. 10A-B, IGFBPL1 deficiency leads to microglia activation and elevated levels of proinflammatory cytokines in the adult retina. FIG. 11 shows that RGC loss and functional deficits in IGFBPL1 deficient mice, which were rescued by IGFBPL1 intravitreal injection.

The present study reveals that IGFBPL1 is expressed by microglia rather than RGCs in adult mice. It exerts a neuroprotective effect by acting on microglia. These results suggest that IGFBPL1 protects from neuronal death through modulating neuroinflammation.

### Example 3. IGFBPL1 Protected Against the Neuronal and Vision Loss in IGFBPL1^{-/-} Mice with Ocular Hypertension

Insulin growth factor binding protein like 1 (IGFBPL1) promotes survival and neurite outgrowth of neonatal mouse retinal ganglion cells (RGC) regulated via insulin like growth factor 1 mediated signaling pathways (Guo et al., Sci Rep. 2018 Feb. 1; 8(1):2054). Neonatal IGFBPL1 deficient (IGFBPL1^{-/-}) mice had approximately 20% less of RGC comparing to wild-type control mice. The present study investigated the role of IGFBPL1 protein on neuronal survival and visual performance of adult IGFBPL1^{-/-} mice with or without elevated intraocular pressure (IOP).

RGC densities of 1, 2 and 7 months-old IGFBPL1−/− mice were determined by Brn3a immunolabeling in retinal flat-mounts. To induce elevation of IOP, two micro liter of polystyrene microbeads (MB; 5×10⁶/ml) were injected into the anterior chamber of unilateral eye of adult male and female IGFBPL1−/− mice. IGFBPL1 recombinant protein or sterile saline as a control was administered by intravitreal injection at 3, 7 and 17 days post-MB injection. At 4 weeks post-MB injection, two investigators recorded the optomotor response of contrast sensitivity and visual acuity of mice in a masked fashion. The mice were sacrificed and the retinas were flat-mounted and processed for Brn3a immunolabeling to reveal surviving RGC. Student's t-test was used for statistical analysis.

The data showed that absence of IGFBPL1 led to progressive loss of RGC in IGFBPL1^{-/-} mice. A transient elevation of IOP produced a significant decline of visual performance and RGC loss in adult IGFBPL1^{-/-} mice. IGFBPL1 treatment significantly improved the visual performance (P<0.05) and RGC survival (P<0.05) in wild-type and IGFBPL1^{-/-} mice with ocular hypertension.

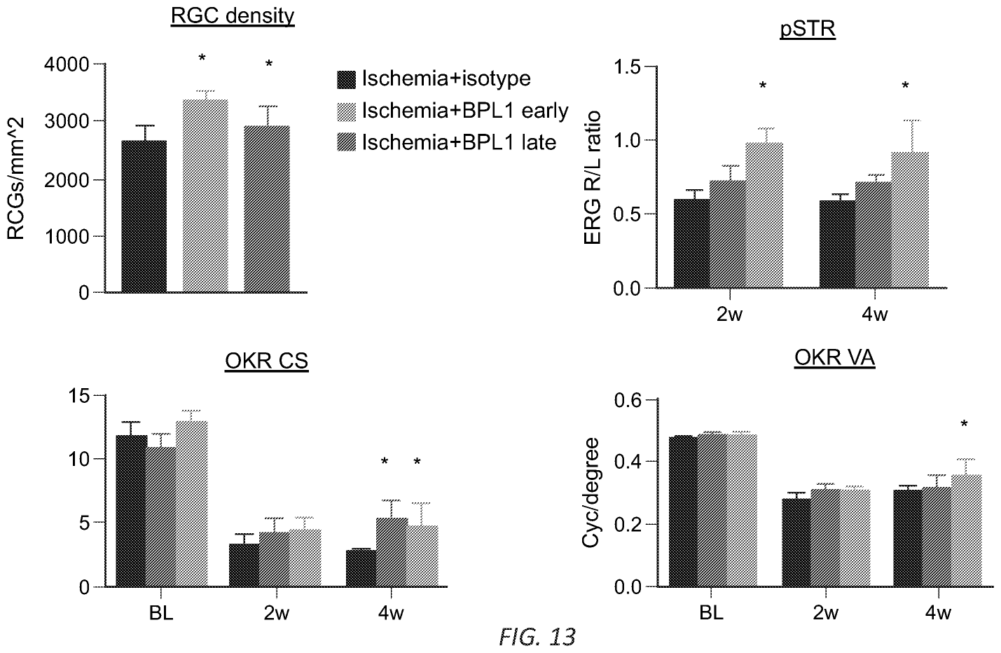

In addition, retinal ischemic reperfusion injury was induced unilaterally in mice, followed by intravitreal injection of saline (isotype) or IGFBPL1 (BPL1) on day 1 (early) or day 10 (late) after injury. Mice were sacrificed 4 weeks after injury and quantified for RGC densities. RGC functions were assessed at 2 and 4 weeks after injury (before sacrifice) by pSTR amplitudes, while visual contrast sensitivity (CS) and visual acuity (VA) were measured using optokinetic reflex (OKR) assays. As shown in FIG. 13, after ischemic injury there was a significant increase of RGC densities and improvement of pSTR amplitudes as well as CS and VA values in IGFBPL1-treated mice compared to saline-treated group.

IGFBPL1 has been known to express strongly in embryonic retina and barely detect in adult retina. These results showed that lack of IGFBPL1 during embryonic stage induces progressive degeneration of RGC in IGFBPL1^{-/-} mice. Administration of IGFBPL1 protected against the RGC and vision loss in mouse with ocular hypertension. Overall, IGFBPL1 is an important neuroprotective agent in retinas undergoing progressive degeneration, such as in glaucoma.

### Example 4. Heat Shock Protein 27 and 60 Directly Activate Human Microglia

Glaucoma has an autoimmune component caused by commensal bacteria-primed CD4+ T cells that enter the retina and cross-react with heat shock protein (HSP)-expressing neurons via a mechanism of molecular mimicry. As shown herein microglial activation is a cause of the immune responses and retinal degeneration in glaucoma. Given the limited availability of primary human microglia, the immortalized human microglia clone 3 cell line (HMC3) is useful for the examination of the microglia behavior under pathological conditions. To test if HSP27 and HSP60 could induce activation of microglia, cytokine expression and morphological changes were examined in HMC3. Other known inflammatory stimulators were used as positive control.

Methods: HMC3 cell line (ATCC) were cultured in EMEM medium and challenged with 10 ug/ml HSP27, 10 ug/ml HSP60, 200 ng/ml LPS or 100 ng/ml LPS with or without 5 mM ATP for additional 30 minutes. Cells received medium alone were used as controls. After 24 hours, RNAs of HMC3 cells were collected by ZYMO Research Quick-RNA Microprep Kit, and RNA reverse transcript was carried out by PrimeScript™ RT Master Mix. Sybr green RT-PCR mixtures containing different primers and cDNA samples were subjected to PCR using EP realplex real-time PCR system. Relative fold changes of mRNA transcripts were presented and compared with the control group. Moreover, low density HMC3 cell cultures were set up, and images of cell morphology were captured 24 hours after LPS, HSP27 or HSP60 treatment, and the morphology changes were quantified.

Results: The data showed that while LPS with or without ATP induced increased expression of pro-inflammatory cytokines such as IFNγ in HMC3, HSP27 and HSP60 could also activate HMC3 to express higher level of IFNγ and TNFα. Quantification of cell morphology showed shortened dendritic processes and enlarged round cell body size in LPS, HSP27 and HSP60 stimulated groups compared to vehicle controls (P<0.05).

Conclusion: The present study revealed that HMC3 cells reacted similarly as primary microglia to the known inflammation stimulators. HSP27 and HSP60 could directly induce pro-inflammatory responses of HMC3 cells, supporting a notion that HSPs may induce microglial activation as an early cause of glaucoma-associated immune responses.

### Example 5. Exploring the Spatial-Temporal Dynamics of Microglia/Macrophage Polarization after Ischemia/Reperfusion in the Retina

Background: Microglia/macrophages exhibit diverse functional phenotypes under various microenvironmental stimulus and disease course. The phenotype-dynamic changes of microglia in ischemia/reperfusion (I/R) remained ambiguous.

Identification of spatial-temporal pattern of microglia/macrophage polarization after I/R may improve our knowledge for post-I/R damage and recovery.

Methods: I/R was induced in rats by cannulating with a 30-gauge needle connected to a normal saline reservoir to maintain an intraocular pressure of 110 mm Hg for 60 min. The retinas of rats were collected at postoperative day 1, 2, 7 and 14. Flow Cytometry, reverse-transcriptase polymerase chain reaction, Western blot and immunohistochemical staining for M1 and M2 markers were performed to characterize phenotypic changes in retinal cells, including microglia and infiltrating macrophages.

Result: Flow cytometry result showed a significant increase of CD11b⁺CD45^{high}, presumably macrophages and/or activated microglia, at as early as 12 hours post I/R, followed by the increase of CD11b⁻CD45^{high} lymphocytes on day 1, both of which were peaked on day 7. A rapid increase of both CD16⁺Iba1⁺ (M1 marker) cell and Ym-1⁺Iba1⁺ (M2 marker) cells were found in the retina of day 1 and day 2 after I/R. These cells exhibited round bodies with scarce short dendrites and distributed from inner nuclear layer to ganglion cell layer at day 1-day 7.

Conclusion: I/R induced an early response of microglia/macrophages that were diversely activated to both M1-type and M2-type, leading to graduate increases of lymphocytes. Thus, microglia/macrophage may play a leading role in the recruitment of infiltrated lymphocytes following I/R.

### Example 6. Targeting HSP: Immune Tolerance to HSP60 Attenuates Neurodegeneration in Glaucoma

Purpose: A previous study suggests that a bacteria-primed T cell-mediated autoimmune mechanism underlies the pathogenesis of glaucoma, and heat shock proteins (HSPs) are thought to act as pathogenic autoantigens. We hypothesized that induction of immune tolerance to bacterial HSP60 might block such a pathogenic immune response and attenuate neuron loss in glaucoma.

Methods: Adult C57BL/6J mice were given a low dose of recombinant bacterial HSP60, Ovalbumin (OVA) or saline (both as controls) in the nostril each day for 7 days. Elevation of IOP was induced unilaterally by an anterior chamber injection of polystyrene microbeads (MB). Visual and retinal functions were assessed by optomotor response (OMR) and electroretinogram positive scotopic threshold response (pSTR). Mice were sacrificed 2, 4 and 8 weeks after MB injection. T cell responses to bacterial HSP60 were analyzed by ear DTH (delayed-type hypersensitivity responses) testing and flow cytometry. Glaucomatous neural damage was quantified by retinal ganglion cell (RGC) and axon counts.

Results: Nostril administration of a low dose HSP60 induced immune tolerance as shown by reduced DTH responses and increased levels of T regulatory cells as seen by analysis of flow cytometry. MB-injected eyes maintained an IOP level of 25±3 mmHg as compared to 12±2 mmHg in the contralateral non-injected eyes. We observed no significant differences in IOP levels between HSP60-, OVA- and saline-treated mice. Treatment with HSP60 or OVA did not alter the basal levels of visual acuity (VA), contrast sensitivity (CS) or pSTR prior to MB-injection, as compared to naïve or saline-treated mice. However, after MB-injection, at all time points, VA and CS as assessed by OMR were significantly better in HSP60-treated mice compared to saline or OVA-treated mice. Consistently, both RGC function assessed by pSTR amplitudes and RGC counts were significantly higher in HSP60-treated mice compared to saline or OVA-treated mice after MB-injection.

Conclusion: Intranasal administration of multiple low doses of bacterial HSP60 induced immune tolerance and attenuated RGC loss and functional deterioration in an MB-induced mouse model of glaucoma, and. These results suggest an attractive antigen-specific therapeutic strategy for the prevention of vision loss in glaucoma. The study may also help in our understanding of the pathogenesis of brain neurodegenerative disorders and provide innovative interventions for the treatment of neurodegeneration affecting other parts of the central nervous system.

### REFERENCES

1. Quigley, H. & Broman, A. T. The number of people with glaucoma worldwide in 2010 and 2020. Br. J. Ophthalmol. 90, 262-267 (2006).
2. Walland, M. J. et al. Failure of medical therapy despite normal intraocular pressure. Clin. Exp. Ophthalmol. 34, 827-836 (2006).
3. Vu, T. H. K., Jager, M. J. & Chen, D. F. The Immunology of Glaucoma. Asia-Pacific J. Ophthalmol. 1, 303-311 (2012).
4. Flammer, J. & Mozaffarieh, M. What Is the Present Pathogenetic Concept of Glaucomatous Optic Neuropathy? Surv. Ophthalmol. 52, 162-173 (2007).
5. Tezel, G. & Wax, M. B. The immune system and glaucoma. Curr. Opin.

Ophthalmol. 15, 80-84 (2004).
6. Wax, M. B. The case for autoimmunity in glaucoma. Exp. Eye Res. 93, 187-190 (2011).
7. Gramlich, O. W. et al. Enhanced Insight into the Autoimmune Component of Glaucoma: IgG Autoantibody Accumulation and Pro-Inflammatory Conditions in Human Glaucomatous Retina. PLoS One 8, 1-11 (2013).
8. Bell, K. et al. Does autoimmunity play a part in the pathogenesis of glaucoma? Prog. Retin. Eye Res. 36, 199-216 (2013).
9. Grus, F. H., Joachim, S. C., Wuenschig, D., Rieck, J. & Pfeiffer, N. Autoimmunity and glaucoma. J. Glaucoma 17, 79-84 (2008).
10. Chen et al., Nat Commun. 2018 Aug. 10; 9(1):3209.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating and/or reducing risk of development or progression of neuroinflammation and neurodegeneration, comprising administration of therapeutically effective amounts of one or more of: (i) HSP60 or HSP27, or active fragments thereof; and/or (iii) IGFBPL or active fragments thereof, to a subject in need thereof.

2. The method of claim 1, wherein the therapeutically effective amounts are sufficient to reduce inflammation and neuronal cell death in the subject.

3. The method of claim 1, comprising nasal or subcutaneous administration of HSP60 and systemic or ocular administration of IGFBPL.

4. The method of claim 3, wherein ocular administration of IGFBPL comprises intravitreal injection.

5. The method of claim 1, wherein the subject has Non-Arteritic Ischemic Optic Neuropathy (NAION), Glaucoma, Autism, Multiple Sclerosis, Alzheimer's Disease, Parkinson's Disease, Ischemic Retinopathy, Age-Related Macular Degeneration, Stroke, Ischemic and Traumatic Optic Neuropathy, or Diabetic Retinopathy.

6. The method of claim 1, wherein the method reduces inflammation and neuronal death in an eye of the subject.

7. A kit comprising: a composition comprising HSP60 and/or HSP27, and a composition comprising IGFBPL1.

8. (canceled)

9. The kit of claim 7, wherein the HSP60 and or HSP27 is formulated for nasal administration and the IGFBPL1 is formulated for ocular administration.

10. The kit of claim 7, wherein the HSP60 and/or HSP27 is formulated for nasal or subcutaneous administration and the IGFBPL1 is formulated for systemic administration.

11. The kit of claim 7, wherein the HSP60 and/or HSP27 is formulated for nasal or subcutaneous administration and the IGFBPL1 is formulated for nasal administration.

12. The kit of claim 11, wherein one, two, or all three of the HSP60 and/or HSP27 and the IGFBPL1 are formulated together for nasal or subcutaneous administration in a single composition.
